(19) United States
(12) Patent Application Publication
(10) Pub. No.: US 2021/0177831 A1
(43) Pub. Date: Jun. 17, 2021

(54) COMPOSITIONS AND METHODS FOR LOCAL DELIVERY OF PHARMACEUTICAL AGENTS TO TREAT CANCER

(71) Applicant: The General Hospital Corporation, Boston, MA (US)

(72) Inventor: Shadmehr Demehri, Charlestown, MA (US)

(21) Appl. No.: 17/257,426

(22) PCT Filed: Jul. 2, 2019
(86) PCT No.: PCT/US19/40343

§ 371 Date: Dec. 31, 2020

Related U.S. Application Data
(60) Provisional application No. 62/694,100, filed on Jul. 5, 2018.
(60) Provisional application No. 62/740,514, filed on Oct. 3, 2018.

(51) Int. Cl. A61K 31/4745; A61K 31/203; A61K 31/593; A61K 31/55; A61K 31/711; A61K 31/353; A61K 31/7084; A61K 38/19; A61K 35/763; A61K 9/00; A61P 35/00

(52) U.S. Cl. CPC A61K 31/4745; A61K 31/203; A61K 31/593; A61K 31/55; A61K 31/711; A61P 35/00; A61K 31/7084; A61K 38/193; A61K 35/763; A61K 9/0014; A61K 31/353

(57) ABSTRACT

Described herein are compositions comprising a T cell/thymic stromal lymphopoietin (TSLP) activating agent and an innate immunity activating agent, an methods of use thereof to treat cancer, e.g., cutaneous malignancies.

### CLAIM OF PRIORITY

This application claims the benefit of U.S. Provisional Application Ser. No. 62/694,100, filed on Jul. 5, 2018; and 62/740,514, filed on Oct. 3, 2018. The entire contents of the foregoing are incorporated herein by reference.

### FEDERALLY SPONSORED RESEARCH OR DEVELOPMENT

This invention was made with government support under Grant No. OD021353 awarded by the National Institutes of Health. The government has certain rights in the invention.

### TECHNICAL FIELD

This invention relates, at least in part, to compositions (e.g., topical compositions) comprising thymic stromal lymphopoietin (TSLP) inducer (e.g., calcipotriene) and an innate immune activating agent (e.g., imiquimod, TLR agonists, STING agonists and oncolytic viruses), and methods of use thereof, e.g., to treat malignancies including skin cancers (e.g., Merkle cell carcinoma, melanoma, extramammary paget's disease, cutaneous T-cell lymphoma (CTCL)), breast cancer (primary and chest wall metastasis), and other cancers.

### BACKGROUND

There are several cutaneous malignancies that are considered poor candidates for surgical excision due to the location of the cancer, the extent of the disease and the morbidities associated with their surgery. These cutaneous malignancies include breast cancer cutaneous metastasis, extramammary Paget's disease and cutaneous melanoma (especially in situ lesions and lentigo maligna)¹⁻³. Systemic treatments and radiation have been used for the treatment of these unresectable cancers with only modest benefit and significant side effects¹⁻³.

### SUMMARY

As shown herein, the combination of one or more T cell/thymic stromal lymphopoietin (TSLP) activating agents (i.e., calcipotriene, retinoic acid) with one or more innate immune cell activating agents (i.e., imiquimod, a STING agonist) leads to a synergistic effect, providing robust antitumor immune activation against cancers, e.g., cancers of the skin including non-keratinocyte cutaneous malignancies (e.g., extramammary Paget's disease and breast metastasis) and melanoma. In particular, topical calcipotriene plus imiquimod, retinoic acid plus imiquimod, and calcipotriene plus DMXAA treatments were significantly more effective than calcipotriol, retinoic acid, imiquimod, or DMXAA monotherapies in blocking breast cancer and melanoma growth. In addition, a short course of topical calcipotriene plus imiquimod treatment was effective for the treatment of extramammary Paget's disease in patients. Thus provided herein are combinations of innate immune activating agents and direct T cell induction by thymic stromal lymphopoietin (TSLP) activating agents, and their use in proliferative disorders, in particular cutaneous malignancies.

Provided herein are methods for treating a proliferative disorder in the skin of a subject in need thereof, comprising administering a therapeutically effective amount of (i) an agent that activates T cells through TSLP induction and (ii) an innate immune cell activating agent, preferably directly to a site of proliferation in the skin of the subject, thereby treating the proliferative disorder in the skin of the subject. Also provided herein is an agent that activates T cells through TSLP induction, and an innate immune cell activating agent method, for use in treating a proliferative disorder in the skin of a subject in need thereof, preferably in the absence of a cytotoxic agent.

In some embodiments, the proliferative disorder in the skin is a non-keratinocyte cancer, e.g., melanoma, extramammary Paget's disease or a breast cancer metastasis.

In some embodiments, the site of proliferation in the skin is a tumor.

In some embodiments, the site of proliferation in the skin comprises a metastasis within the chest wall of the subject.

In some embodiments, the method does not include administering a cytotoxic agent.

In some embodiments, the agent that activates T cells through TSLP induction is a Vitamin D analog, e.g., calcipotriene, or retinoic acid.

In some embodiments, the innate immune cell activating agent is a TLR agonist, STING agonist, or oncolytic virus.

In some embodiments, the TLR agonist is imiquimod, resiquimod, 852A, motolimod (VTX-2337); CpG-ODN; or ODN2395.

In some embodiments, the STING agonist is DMXAA, MK-1454 or ADU-S100.

In some embodiments, the oncolytic virus is Talimogene Laherparepvec (T-Vec).

In some embodiments, a composition comprising an agent that activates T cells through TSLP induction and a composition comprising an innate immune cell activating agent are administered concomitantly or sequentially.

In some embodiments, a single composition comprising an agent that activates T cells through TSLP induction and an innate immune cell activating agent is administered.

In some embodiments, one or both of the agent that activates T cells through TSLP induction and the innate immune cell activating agent is topically administered.

Also described herein are methods for reducing or eliminating a proliferative disorder in the skin of a subject in need thereof, said methods comprising topically administering a composition comprising Calcipotriene and Imiquimod to a site of proliferation in the skin of the subject, thereby reducing or eliminating the proliferative disorder in the skin of the subject, and wherein the proliferative disorder of the skin does not comprise a keratinocyte carcinoma.

In some embodiments, the proliferative disorder in the skin is a cutaneous malignancy.

In some embodiments, the cutaneous malignancy is melanoma, extramammary Paget's disease or breast cancer.

In some embodiments, the site of proliferation in the skin is a tumor.

In some embodiments, the tumor weight (size) is synergistically reduced.

In some embodiments, the site of proliferation in the skin comprises a metastasis within the chest wall of the subject.

In some embodiments, the keratinocyte carcinoma is a Squamous Cell Carcinoma (“SCC”), Basal Cell Carcinoma (“BCC”), or a keratinocyte cancer.

In some embodiments, the composition comprising Calcipotriene and Imiquimod further comprises retinoic acid.

In addition, described herein are methods for reducing or eliminating a proliferative disorder in the skin of a subject in need thereof, said methods comprising topically administering a composition consisting essentially of Calcipotriene and Imiquimod to a site of proliferation in the skin of a subject diagnosed with melanoma, extramammary Paget's disease or breast cancer, thereby reducing or eliminating the proliferative disorder in the skin of the subject.

Additionally described herein are methods for reducing or eliminating a proliferative disorder in the skin of a subject in need thereof, said methods comprising topically administering a composition comprising Calcipotriene and a composition comprising Imiquimod to a site of proliferation in the skin of the subject, thereby reducing or eliminating the proliferative disorder in the skin of the subject, and wherein the proliferative disorder of the skin does not comprise a keratinocyte carcinoma.

In some embodiments, the composition comprising Calcipotriene and the composition comprising Imiquimod are administered concomitantly or sequentially.

In some embodiments, a composition comprising retinoic acid is administered, either concomitantly or sequentially with the composition comprising Calcipotriene and the composition comprising Imiquimod.

Further described herein are methods for reducing or eliminating a proliferative disorder in the skin of a subject in need thereof, said method comprising locally administering Calcipotriene and a STING agonist to a site of proliferation in the skin of the subject, thereby reducing or eliminating the proliferative disorder in the skin of the subject.

In some embodiments, Calcipotriene is topically administered.

In some embodiments, the proliferative disorder in the skin is a cutaneous malignancy.

In some embodiments, the cutaneous malignancy is melanoma, extramammary Paget's disease or breast cancer.

In some embodiments, the site of proliferation in the skin is a tumor.

In some embodiments, the tumor weight is synergistically reduced.

In some embodiments, the site of proliferation in the skin comprises a metastasis within the chest wall of the subject.

In some embodiments, the methods include topical application of a composition comprising 0.001-0.01%, e.g., 0.005%, calcipotriene or another Vitamin D analog, and a composition comprising 1-10%, e.g., 5%, imiquimod or another TLR agonist, or retinoic acid; in some embodiments, a single composition comprising 0.001-0.01%, e.g., 0.005%, calcipotriene or another Vitamin D analog, and 1-10%, e.g., 5%, imiquimod or another TLR agonist, is used.

In some embodiments, a composition as described herein is an ointment, salve, gel, or cream.

In some embodiments, where two compositions are used, one composition is applied topically (e.g., the TSLP inducer) and the other composition is administered by injection, e.g., intralesional/intratumoral injection (e.g., a STING agonist).

### Definitions

Unless otherwise defined, all technical and scientific terms used herein have the same meaning as commonly understood by one of ordinary skill in the art to which this invention belongs. In case of conflict, the present application, including definitions will control.

As used herein, a “subject” is a vertebrate, including any member of the class mammalia, including humans, domestic and farm animals, and zoo, sports or pet animals, such as mouse, rabbit, pig, sheep, goat, cattle and higher primates.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

As used here in, reference to synergy or synergistic effects, including a “synergistic reduction in tumor weight” refers to an effect that is greater than the additive effect expected from using two or more therapeutic agents in combination.

As used herein a “reduction” or “reducing” is by an amount that is at least about 0.05 fold less (for example 0.1, 0.2, 0.3, 0.4, 0.5, 1, 5, 10, 25, 50, 100, 1000, 10,000-fold or more less) than an untreated control or reference standard. A “reduction” or “reducing” can also refer to an amount that is at least about 5% less (for example 5, 6, 7, 8, 9, 10, 15, 20, 25, 30, 35, 40, 45, 50, 55, 60, 65, 70, 75, 80, 85, 90, 95, 99 or 100% or more less) than an untreated control or reference standard.

Unless specifically stated or clear from context, as used herein, the term “about” is understood as within a range of normal tolerance in the art, for example within 2 standard deviations of the mean. “About” is understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Other definitions appear in context throughout this disclosure.

Other features and advantages of the invention will be apparent from the Detailed Description, and from the claims. Thus, other aspects of the invention are described in the following disclosure and are within the ambit of the invention.

### BRIEF DESCRIPTION OF THE DRAWINGS

The following Detailed Description, given by way of example, but not intended to limit the invention to specific embodiments described, may be understood in conjunction with the accompanying figures, incorporated herein by reference.

FIGS. 1A-1F show that calcipotriene plus imiquimod blocked breast cancer growth. n=4 per group; Error bars represent the mean+SD; two-tailed Mann-Whitney U test is used as the test of significance; scale bar: 1 cm.

(1A) Schematic diagram of the experimental protocol used to determine the efficacy of calcipotriene plus imiquimod as a topical immunotherapy for breast cancer. PyMTtg primary breast cancer cells are implanted subcutaneously into the inguinal regions of wild-type C57BL/6 mice. When tumors reached 5 mm in diameter (day 13), the following topical treatments applied to the tumor sites twice: (a) 80 nmol calcipotriene in 20 microliters of 100% EtOH followed by 5% imiquimod cream, (b) 80 nmol calcipotriene in 20 microliter 100% EtOH followed by moisturizing (control) cream, (c) 20 microliter 100% EtOH followed by 5% imiquimod cream, and (d) 20 microliter 100% EtOH followed by moisturizing (control) cream.

(1B) Macroscopic images of representative breast tumors in each treatment group at the endpoint show significantly smaller tumor in calcipotriene plus imiquimod-treated mouse compared to mice in the other groups.

(1C) Tumor volume measurements over time are shown. Tumor growth is blocked upon calcipotriene plus imiquimod treatment (p=0.011). Arrows highlight the treatment timepoints.

(1D) The endpoint tumor weights are shown. Breast tumors treated with calcipotriene plus imiquimod are significantly smaller than those in the other treatment groups (*: p<0.05).

(1E) Flow cytometric analysis of breast tumors show induction of T cell (CD3+) in the calcipotriene plus imiquimod-treated tumors.

(1F) CD4+ and CD8+ T cells in calcipotriene plus imiquimod-treated tumors are highly proliferative (Ki67+) compared to T cells isolated from the tumors of other treatment cohorts.

FIGS. 2A-F show that Calcipotriene plus STING agonist (DMXAA) blocked melanoma growth. n=3 per group; Error bars represent the mean+SD; two-tailed Mann-Whitney U test is used as the test of significance; scale bar: 1 cm.

(2A) Schematic diagram of the experimental protocol used to determine the efficacy of calcipotriene plus DMXAA as a localized immunotherapy for melanoma. B16-F10 melanoma cells are implanted subcutaneously into the flanks of wild-type C57BL/6 mice. When tumors reached 5 mm in diameter (day 14), the following treatments were applied three times: (a) 40 nmol calcipotriene in 20 microliters of 100% EtOH followed by IP injection of 400 g DMXAA in 100 saline, (b) 40 nmol calcipotriene in 20 microliters of 100% EtOH followed by IP injection of 100 saline, (c) 20 microliters of 100% EtOH followed by IP injection of 400 g DMXAA in 100 saline, and (d) 20 microliter 100% EtOH followed by IP injection of 100 saline.

(2B) Macroscopic images of representative melanoma tumors in each treatment group at the endpoint show significantly smaller tumor in calcipotriene plus DMXAA-treated mouse compared to mice in the other groups.

(2C) Tumor volume measurements over time are shown. Tumor growth is blocked upon calcipotriene plus DMXAA treatment (p=0.019). Arrows highlight the treatment timepoints.

(2D) The endpoint tumor weights are shown. Melanoma tumors treated with calcipotriene plus DMXAA are significantly smaller than those in the other treatment groups (*: p<0.05).

(2E) Flow cytometric analysis of melanoma tumors show induction of T cell (CD3+) in the calcipotriene plus DMXAA-treated tumors.

(2F) CD4+ and CD8+ T cells in calcipotriene plus DMXAA-treated tumors are highly proliferative (Ki67+) compared to T cells isolated from the tumors of other treatment cohorts.

FIGS. 3A-D show that Calcipotriene plus imiquimod and retinoic acid plus imiquimod blocked melanoma growth. n=3 per group; Error bars represent the mean+SD; two-tailed Mann-Whitney U test is used as the test of significance; scale bar: 1 cm.

(3A) Schematic diagram of the experimental protocol used to determine the efficacy of calcipotriene plus imiquimod and retinoic acid plus imiquimod as localized combination immunotherapy for melanoma. B16-F10 melanoma cells are implanted subcutaneously into the flanks of wild-type C57BL/6 mice. When tumors become palpable (day 4), the following treatments applied three times: (a) 20 nmol calcipotriene in 20 microliters of 100% EtOH followed by 5% imiquimod cream, (b) 20 nmol retinoic acid in 20 microliters of 100% EtOH followed by 5% imiquimod cream, (c) 20 nmol calcipotriene in 20 microliter 100% EtOH followed by moisturizing (control) cream, (d) 20 nmol retinoic acid in 20 microliter 100% EtOH followed by control cream, and (e) 20 microliter 100% EtOH followed by 5% imiquimod cream.

(3B) Macroscopic images of representative melanoma tumors in each treatment group at the endpoint show significantly smaller tumor in calcipotriene plus imiquimod and retinoic acid plus imiquimod-treated mice compared to mice in the monotherapy groups.

(3C) Tumor volume measurements over time are shown. Tumor growths are blocked upon calcipotriene plus imiquimod and retinoic acid plus imiquimod treatment compared to imiquimod monotherapy treatment (p=0.05). Arrows highlight the treatment timepoints (ns: not significant).

(3D) The endpoint tumor weights are shown. Melanoma tumors treated with calcipotriene plus imiquimod and retinoic acid plus imiquimod are significantly smaller than those in the other treatment groups (*: p<0.05; ns: not significant).

FIGS. 4A-B show that Calcipotriene plus imiquimod combination reduced EMPD lesion size in patients.

(4A) Schematic diagram of the 4-day calcipotriene plus imiquimod treatment used to treat two EMPD patients.

(4B) Macroscopic images of EMPD lesions on the genitals of the two patients pre and post-treatment. Note that the “After treatment” images are taken at the time of surgery. Yellow dashed lines highlight the margins of the lesions before and after treatment.

### DETAILED DESCRIPTION

The present findings demonstrated that the combination of an agent that activates T cells through TSLP induction (e.g., calcipotriene or retinoic acid) with an innate immune cell activating agent (e.g., imiquimod, TLR agonists, STING agonists, or oncolytic viruses) can yield a synergic efficacy against cancer. These combinations lead to an optimal activation of the adaptive immunity against malignancies including breast cancer cutaneous lesion, extramammary Paget's disease, melanoma, and other lesions that are currently being treated with imiquimod. These findings also provide a treatment modality to transform cold (nonimmunogenic) cancers to hot (inflamed) cancers by combining the innate signaling in the myeloid cells with direct T cell stimulation by TSLP to induce robust immunity against the cancer. These combination therapies destroy the cancer cells (target) by inducing optimal antigen presentation by the dendritic cells and macrophages (imiquimod/DMXAA effect) and inducing full T cell activation upon detection of the tumor-associated antigens (calcipotriene/retinoic acid effect). These combinations, which target signal 1, signal 2, and signal 3 for full T cell activation, lead to robust antitumor adaptive immunity and an effective cancer treatment. Although these combinations can be developed to treat internal cancers through their delivery into the tumor, the capability to deliver these agents in a topical combination creates a unique opportunity to effectively treat cutaneous malignancies like melanoma, extramammary Paget's disease and cutaneous metastasis from breast and other internal cancers. Among these malignancies, extramammary Paget's disease is a rare adenocarcinoma of apocrine gland-bearing skin (with a similar developmental origin as breast gland/cancer)². This cancer is notoriously difficult to manage². Surgery in these patients carries a high recurrence rate of 30% and causes significant morbidity due to the location of the lesions that are mostly in the groin and genitals^{13,14}. In addition, there is a limited data on the efficacy of chemotherapy and radiation for this cancer¹⁵. Therefore, the present combinations can be used as effective therapeutic approaches for extramammary Paget's disease, which may revolutionize the care of patients with this debilitating disease.

### Methods of Treatment

The methods described herein include methods for the treatment of proliferative disorders, in particular cutaneous malignancies. Generally, the methods include administering a therapeutically effective amount of a combination therapy as described herein, i.e., comprising an agent that activates T cells through TSLP induction (e.g., calcipotriene or retinoic acid) and an innate immune cell activating agent (e.g., imiquimod, TLR agonists, STING agonists, or oncolytic viruses), to a subject who is in need of, or who has been determined to be in need of, such treatment.

Generally speaking, the present compositions and methods do not include the use of cytotoxic agents, e.g., as described in US2017/0246299.

As used in this context, to “treat” means to ameliorate at least one symptom of the proliferative disorder. Administration of a therapeutically effective amount of a compound described herein for the treatment of a proliferative disorder will result in decreased lesion or tumor growth rate, lesion or tumor regression, and/or reduced lesion or tumor size, and may increase lifespan, reduce risk of recurrence, and/or reduce the need for surgical intervention.

The combinations described herein are useful in the treatment of disorders associated with abnormal apoptotic or differentiative processes, e.g., cellular proliferative disorders or cellular differentiative disorders, e.g., cancer, e.g., by producing an active immunity. Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, rectum, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

In some embodiments, the disorder is a cutaneous non-keratinocyte cancer, e.g., extramammary Paget's disease, breast cancer cutaneous metastasis, and cutaneous melanoma (e.g., in situ lesions and lentigo maligna).

In some embodiments, the disorder is not a keratinocyte carcinoma, i.e., is not basal-cell carcinoma or cutaneous squamous cell carcinoma (SCC).

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the disease is cutaneous T-cell lymphoma (CTCL).

In some embodiments, the proliferative disorders include pre-cancerous skin disorders. The skin disorder may involve the aberrant activity of a cell or a group of cells or layers in the dermal, epidermal, or hypodermal layer, or an abnormality in the dermal-epidermal junction.

Thymic Stromal Lymphopoietin (TSLP) Inducing Agents

The present combinations include the use of TSLP inducing agents. As used herein, a “TSLP inducer” is any compound that is capable of inducing thymic stromal lymphopoietin (TSLP). TSLP is an epithelial-derived cytokine that belongs to the interleukin-7 (IL-7) cytokine family. A number of such agents are known in the art. Methods to determine if a compound induces TSLP are known in the art. For example, TSLP nucleic acid expression, TSLP protein expression, or TSLP activity may be measured as described in more detail in US2017/0246299, which is incorporated herein in its entirety. Non-limiting examples of TSLP inducers include retinoic acid, vitamin D analogs, polyinosinic-polycytidylic acid (poly(I.C) and other TLR3 ligands), FSL-1 (and other TLR2-TLR6 ligand), flagellin (and other TLRS ligand), beta2-adrenoceptor agonists, cAMP-elevating agents (e.g., Forskolin), fatty acids (heptanoic acid, octanoic acid, nonanoic acid, and decanoic acid), xylene, 1,2,4-trimethylbenzene, 12-O-tetradecanoyl phorbol-13-acetate (TPA, tetradecanoylphorbol acetate, tetradecanoyl phorbol acetate, and phorbol 12-myristate 13-acetate (PMA)), dibutyl phthalate (DBP), and diisononyl phthalate (DINP).

Retinoic acid is a metabolite of vitamin A (retinol) and an inducer of TSLP. It is known as Tretinoin, Vitamin A acid, ATRA, and all-trans-Retinoic acid.

In some embodiments, a TSLP inducer may be vitamin D or an analog thereof. Vitamin D refers to a group of fat-soluble secosteroids. Secosteroids are very similar in structure to steroids except that two of the B-ring carbon atoms of the typical four steroid rings are not joined, whereas in steroids they are. Examples include active vitamin D (or calcitriol), and Vitamin D₃, also known as cholecalciferol (a form of vitamin D generated in the skin of animals when light energy is absorbed by a precursor molecule 7-dehydrocholesterol).

Structurally modified derivatives of vitamin D may be referred to as vitamin D analogs. Vitamin D analogs may be modified to improve bioavailability, solubility, have improved stability and/or handling properties compared to an unmodified version. Prodrugs of vitamin D analogs are also contemplated. Any vitamin D analog capable of binding to vitamin D receptor and inducing thymic stromal lymphopoietin (TSLP) may be suitable for a composition as described herein. Vitamin D analogs are many, and will be recognized by the skilled person. Non-limiting examples of suitable vitamin D analogs include I, 24-(OH)₂D₃ (calcitrol), 26,27-F_{fl-I}, 25-(OH)₂D₃ (ST-630), .alpha.-(OH)D₂, Iα-(OH)D₃, I,24-(OH)₂D₃ (TV-02), 22-oxacalcitriol (OCT), calcipotriol (MC 903), I,25-(OH)₂-16-ene-23-yne-D₃ (Ro 23-7553), EB 1089, ED-71, PRI-2191, PRI-2205, cholecalciferol, ergocalciferol, calciferol, Calcijex, calcitriol, doxercalciferol, Hectorol, paricalcitol, Rocaltrol, Daivonex, and Zemplar, and other analogs, e.g., as described in e.g., as described in U.S. Pat. No. 6,753,013. The term “analog”, in the context of the present invention, is meant to include synthetic analogs as well as vitamin D metabolites.

In some embodiments, the vitamin D analog is calcipotriol (also known as calcipotriene, or calcitrene). Calcipotriene is a synthetic derivative of calcitriol, a form of vitamin D. It is used in the treatment of psoriasis. Leo Pharma secured FDA approval in 1993 for Calcipotriene topical ointment. Generic versions have been approved (GlenMark and Tolmar). The induction of an epidermis-derived cytokine, thymic stromal lymphopoietin (TSLP), protects against skin and breast cancer development⁷⁻⁹. This protection is mediated by T cells responding directly to TSLP^{7,8}. Calcipotriene, a low-calcemic vitamin D analog and inducer of TSLP, is an immunotherapy that has been used for breast cancer treatment⁸.

Innate Immune Activating Agents

The present combinations also include the use of innate immune activating agents that activate antigen presenting cells. Activators of the innate immune system include agonists of the toll-like receptors (TLRs); STING agonists; or oncolytic viruses.

A number of TLR agonists, e.g., agonists or activators of TLR7, TLR8, or TLR9, are known in the art including imiquimod (TLR7), resiquimod (TLR7), 852A ((N-[4-(4-amino-2-ethyl-1H-imidazo[4,5-c]quinolin-1-yl) butyl]methanesulfonamide) (TLR7); motolimod (VTX-2337) (TLR8); CpG-ODN (TLR9), ODN2395 (TLR9); other TLR agonists that can be used include BCG (TLR 2/4/9); Poly I:C/PolyICLC (TLR3); or MPL (TLR4). See, e.g., Mifsud et al., Front Immunol. 2014; 5:79; Adams, Immunotherapy. 2009 Nov. 1; 1(6): 949-964; Kaczanowska et al., J Leukoc Biol. 2013 June; 93(6): 847-863. In some embodiments, the TLR agonist is not LPS. Imiquimod is a prescription medication that acts as an immune response modifier and is used to treat genital warts, superficial basal cell carcinoma, and actinic keratosis. 3M obtained the first FDA approval in 1997 for Imiquimod under the brand Aldara. As of 2015, Imiquimod is generic and is available worldwide under many brands (Medicis, Apotex, Fougera, Glenmark, Perrigo Israel, Strides, Taro, and Tolmar). The only topical agent that has been used experimentally to assess potential as a treatment of breast cancer, cutaneous metastasis, extramammary Paget's disease and melanoma is Imiquimod^{1,4-6}. Imiquimod is a toll-like receptor (TLR) 7 agonist that works through the activation of the innate immune responses in the skin but does not activate adaptive immune cells directly⁴.

As used herein, a “STING agonist” is a synthetic cyclic dinucleotide (CDN) and other agonist of stimulator of interferon genes protein (STING), which can activate cGAS-STING cytosolic DNA sensing pathway within the cells leading to the activation of the innate immune cells like dendritic cells (e.g., DMXAA, MK-1454 and ADU-S100). DMXAA (5,6-Dimethylxanthenone-4-acetic Acid, ASA404, or Vadimezan) is a STING agonist that functions as a vascular disrupting agent. In some embodiments, where a STING agonist is used, it is locally administered, e.g., by intralesional/intratumoral injection, e.g., as is known in the art.

Oncolytic viruses (OVs) can replicate in cancer cells but not in normal cells, leading to lysis of the tumor mass and triggering of the innate immune system. OVs include Talimogene Laherparepvec (T-Vec) (Amgen); TBI-1401(HF10) (Takara); G207 (MediGene); HSV1716 (Virtu Biologics); ADV/HSV-tk (Merck); LOAd703 (Lokon); CG0070 (Cold Genesys); ColoAd1(Enadenotucirev) (PsiOxus); ONCOS-102 (Targovax Oy); DNX-2401 (DNAtrix); VCN-01 (VCN); Ad-MAGEA3 and MG1-MAGEA3 (Turnstone); NSC-CRAd-Survivin-pk7 (Northwestern); Ad5-yCD/mutTKSR39rep-hIL12 (Henry Ford); Ad5-yCD/mutTKSR39rep-ADP (Henry Ford); MV-NIS (Mayo); MV-NIS (University of Arkansas); GL-ONC1 (Genelux); Pexastimogene Devacirepvec (Pexa-Vec) (Jennerex); REOLYSIN (Oncolytics); CVA21(CAVATAK) (Viralytics); H-1PV(ParvOryx) (Oryx GmbH); PVSRIPO (Duke); and VSV-hIFNbeta-NIS (Mayo). See, e.g., Raja et al., Journal for ImmunoTherapy of Cancer (2018) 6:140; Marelli et al., Front Immunol. 2018; 9: 866.

### Pharmaceutical Compositions and Methods of Administration

The methods described herein include the use of pharmaceutical compositions comprising one or both of an agent that activates T cells through TSLP induction and an innate immune cell activating agent as an active ingredient. The methods can include the use of a single composition including both agents, or two compositions each comprising one of the agents. In some embodiments, these are the only active agents, i.e., no other active agents are included in the compositions or methods.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions; in some embodiments, however, no other active compounds are used.

The present compositions and methods do not include the use of cytotoxic agents, e.g., as described in US2017/0246299, and/or do not include the use of anti-inflammatory drugs, e.g., NSAIDS such as diclofenac, and/or do not include the use of steroidal compounds, e.g., hydrocortisone valerate.

Pharmaceutical compositions are typically formulated to be compatible with its intended route of administration. Examples of routes of administration include parenteral, e.g., intravenous, intradermal, intralesional/intratumoral, intrasubcutaneous; transdermal or topical; transmucosal; or rectal or vaginal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

In some embodiments, where the TLSP inducer is calcipotriol or another vitamin D analog, and the methods include topical application, a solvent is also included that promotes stability of the vitamin D/D analog, e.g., as described in U.S. Pat. No. 6,753,013, non-limiting examples of which include: Arlamol E (polyoxyethylene(15) stearyl ether); Arlamol DoA (diisooctyl ester of adipic acid); Arlasolve 200 (Polyoxyethylene-20-isohexadecyl ether); Eutanol G (2-octyldodecanol); Finsolv (Isostearyl benzoate); Finsolv P (polyoxypropylene-I5-stearyl ether benzoate); Isopropylesters of straight or branched C.sub.10-C.sub.18 alkanoic or alkenoic acids such as isopropyl myristate, isopropyl palmitate, isopropyl isostearate, isopropyl linolate and isopropyl monooleate; Miglyol 840 (Propylene glycol diester of caprylic and caprinic acid); DPPG (propylene glycol dipelagonate); and Procetyl AWS.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For topical or transdermal administration, the active compounds are formulated into ointments, salves, gels, or creams as generally known in the art.

In some embodiments, compositions comprising one or both agents for topical application can further comprise cosmetically-acceptable carriers or vehicles and any optional components. A number of such cosmetically acceptable carriers, vehicles and optional components are known in the art and include carriers and vehicles suitable for application to skin (e.g., ointments, sunscreens, creams, milks, lotions, masks, serums, etc.), see, e.g., U.S. Pat. Nos. 6,645,512 and 6,641,824. In particular, optional components that may be desirable include, but are not limited to absorbents, anti-acne actives, anti-caking agents, anti-cellulite agents, anti-foaming agents, anti-fungal actives, anti-inflammatory actives, anti-microbial actives, anti-oxidants, antiperspirant/deodorant actives, anti-skin atrophy actives, anti-viral agents, anti-wrinkle actives, artificial tanning agents and accelerators, astringents, barrier repair agents, binders, buffering agents, bulking agents, chelating agents, colorants, dyes, enzymes, essential oils, film formers, flavors, fragrances, humectants, hydrocolloids, light diffusers, nail enamels, opacifying agents, optical brighteners, optical modifiers, particulates, perfumes, pH adjusters, sequestering agents, skin conditioners/moisturizers, skin feel modifiers, skin protectants, skin sensates, skin treating agents, skin exfoliating agents, skin lightening agents, skin soothing and/or healing agents, skin thickeners, sunscreen actives, topical anesthetics, vitamin compounds, and combinations thereof. In some embodiments, the compositions include one or more of benzyl alcohol, cetyl alcohol, glycerin, isostearic acid, methylparaben, polysorbate 60, propylparaben, purified water, sorbitan monostearate, stearyl alcohol, white petrolatum, and/or xanthan gum.

The compositions can also be prepared in the form of suppositories (e.g., with conventional suppository bases such as cocoa butter and other glycerides) or retention enemas for rectal or vaginal delivery. Such suppositories can be used particularly for the treatment of conditions associated with lesions or tumors present in the reproductive or gastrointestinal tract.

The pharmaceutical compositions can be included in a container, pack, or dispenser together with instructions for administration.

Topical compositions comprising one or both of an agent that activates T cells through TSLP induction and an innate immune cell activating agent, e.g., Imiquimod and/or Calcipotriene and/or retinoic acid, are suitable for safe and effective use within a range of concentrations. Accordingly, the amount of topical active agent in compositions described herein is between 5 ug/mL and 2000 ug/mL, for example, 25 ug/mL, 100 ug/mL, 500 ug/mL 1000 ug/mL, 1500 ug/mL or 2000 ug/mL. In specific embodiments, calcipotriene (0.005% ug/mL) and about 5% ug/mL to about 3.75% ug/mL imiquimod are used in combination. In other specific embodiments, retinoic acid is administered topically in doses of 0.01%, 0.025%, 0.04%, 0.05% or 0.1% in a cream or gel form. The dosage schedule for this use, i.e., the dosing regimen, will depend upon a variety of factors, including the stage of the disease or condition, the severity of the disease or condition, the general state of the patient's health, the patient's physical status, age and the like. At a minimum, dosing should continue for at least 1 month.

The topical compositions should also be suitable for safe and effective use within a pH range. Accordingly, the pH of topical compositions described herein is between 6.0 and 7.5.

In some embodiments, formulations as described herein are suitable for topical administration. Methods as described herein are ideally suited for increasing an amount of at least one agent in the epidermis, or epidermis and dermis, within a region of a subject's skin. Topical compositions described herein can be administered in dosages suitable to enter the blood stream. Accordingly, agents can be administered topically, as adjuvants, prior to, or concomitantly with, systemic administration directly into the blood stream.

The formulations may conveniently be presented in unit dosage form and may be prepared by any methods well known in the art of pharmacy. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, ointments, pastes, emulsions, sprays, creams, lotions, slurries, suspensions, foams, controlled release formulations, gels, patches, implants, water-oil bilayer compositions, water-oil-powder trilayer compositions, serums, powders, mousses, hydrogels, single-use applicators, and the like, suitable for topical administration to the patient.

The formulations described herein can include a dermatologically acceptable carrier (also referred to herein simply as a “carrier”) for the composition. The phrase “dermatologically acceptable carrier”, as used herein, means that the carrier is suitable for topical application to the hair/scalp, has good aesthetic properties, is compatible with the ETA in the composition, and will not cause any unreasonable safety or toxicity concerns. A suitable carrier is selected to yield a desired product form. Furthermore, the solubility or dispersibility of the components may dictate the form and character of the carrier. In some embodiments, the carrier is present at a level of from about 50 wt % to about 99 wt %, about 60 wt % to about 98 wt %, about 70 wt % to about 98 wt %, or, alternatively, from about 80 wt % to about 95 wt %, by weight of the composition.

The carrier can be in a wide variety of forms. Non-limiting examples include simple solutions (e.g., aqueous, organic solvent, or oil based), emulsions, and solid forms (e.g., gels, sticks, flowable solids, or amorphous materials). In certain embodiments, the dermatologically acceptable carrier is in the form of an emulsion. Emulsion may be generally classified as having a continuous aqueous phase (e.g., oil-in-water and water-in-oil-in-water) or a continuous oil phase (e.g., water-in-oil and oil-in-water-in-oil). The oil phase of the present invention may comprise silicone oils, non-silicone oils such as hydrocarbon oils, esters, ethers, and the like, and mixtures thereof.

The aqueous phase comprises water, such as demineralized or distilled water, for example. Other acceptable carriers that may be used in the aqueous carrier include, but are not limited to alcohol compounds, such as ethanol. According to some embodiments, the composition comprises alcohol, dipropylene glycol, and/or water.

Emulsions may further comprise an emulsifier. The composition may comprise any suitable percentage of emulsifier to sufficiently emulsify the carrier. Suitable weight ranges include from about 0.1 wt % to about 10 wt % or about 0.2 wt % to about 5 wt % of an emulsifier, based on the weight of the composition. Emulsifiers may be nonionic, anionic, or cationic. Suitable emulsifiers are disclosed in, for example, U.S. Pat. Nos. 3,755,560 and 4,421,769, and McCutcheon's Detergents and Emulsifiers, North American Edition, pages 317-324 (1986), which are incorporated herein by reference in their entirety. Suitable emulsions may have a wide range of viscosities, depending on the desired product form. Non-limiting examples of emulsifiers include glyceryl stearate, polysorbate 60, and the PEG-6/PEG-32/glycerol stearate mixture sold under the name of Trefose® by Gattefosse. An emulsion may contain a fatty phase that may range from between about 5 wt % to about 80 wt % (e.g., between about 5 wt % to about 50 wt %) of the composition. Any of the emulsions described herein may contain one or more agents selected from the group of oils, waxes, emulsifiers, and coemulsifiers. Examples of oils, waxes, emulsifiers, and coemulsifiers used in formulations are well-known in the art. An emulsifier and a coemulsifier may be present in the composition in a proportion ranging from 0.3 wt % to about 30 wt % (e.g., between about 0.5 wt % to about 20 wt %) of the composition. An emulsion may contain lipid vesicles.

Topical compositions can be formulated together with other agents, including, but not limited to, other agents for the treatment of skin cancer, inflammatory diseases, benign neoplasms (e.g. hemangiomas), pigmentary disorders, diagnostic agents and cosmetic agents. Such formulations can be configured to control release of the multiple agents at different rates, either sequentially or concomitantly.

Other agents that can be administered together with, or following administration, include but are not limited to, antimicrobial agents, antiseptic agents and analgesic agents. Such agents can be administered sequentially (e.g., after prior administration of topical compositions) or concomitantly, for example, in a dual release dosage formulation.

In some embodiments, the methods include administration of a treatment described herein daily, e.g., once or twice daily, for at least 2, 3, 4, 5, 6, 7, 8 9, 10, 11, 12, 13 or 14 days, e.g., for 4 days, before surgical excision of the remaining lesion, e.g., at 7, 10, 12, 14, 21, or 28, e.g., 7-28, 14-21, days post treatment. In some embodiments, the methods include repeating the treatment regimen at least two, three, four, five, six or more times before optional surgical intervention. In some embodiments, a treatment regimen includes a treatment interval including administration of a treatment described herein daily, e.g., once or twice daily, for at least 2, 3, 4, 5, 6, 7, 8 9, 10, 11, 12, 13 or 14 days, e.g., for 4 days, and then a resting interval, e.g., of at least 7, 10, 12, 14, 21, or 28, e.g., 7-28, 14-21, days, between treatment intervals.

The present invention is additionally described by way of the following illustrative, non-limiting Examples that provide a better understanding of the present invention and of its many advantages.

### EXAMPLES

The following Examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following Examples do not in any way limit the invention.

### Example 1. Compositions Comprising Calcipotriene and Imiquimod Exhibit Anti-Tumor Efficacy

The present inventors hypothesized that the combination of a T cell activating agent (e.g., calcipotriene and retinoic acid 10) with an innate immune cell activating agent (e.g., TLR agonists like imiquimod and STING agonists like DMXAA11) could lead to robust antitumor immunity against cutaneous malignancies. To test this hypothesis, the efficacy of calcipotriene plus imiquimod, calcipotriene plus DMXAA and retinoic acid plus imiquimod was investigated for efficacy in inhibiting cutaneous breast cancer and melanoma growth.

To test the hypothesis that the combination of a T cell activating agent (i.e., calcipotriene) with an innate immune cell activating agent (i.e, imiquimod) could lead to robust antitumor immunity against cutaneous malignancies, the efficacy of calcipotriene plus imiquimod in inhibiting cutaneous breast cancer growth in mice was investigated.

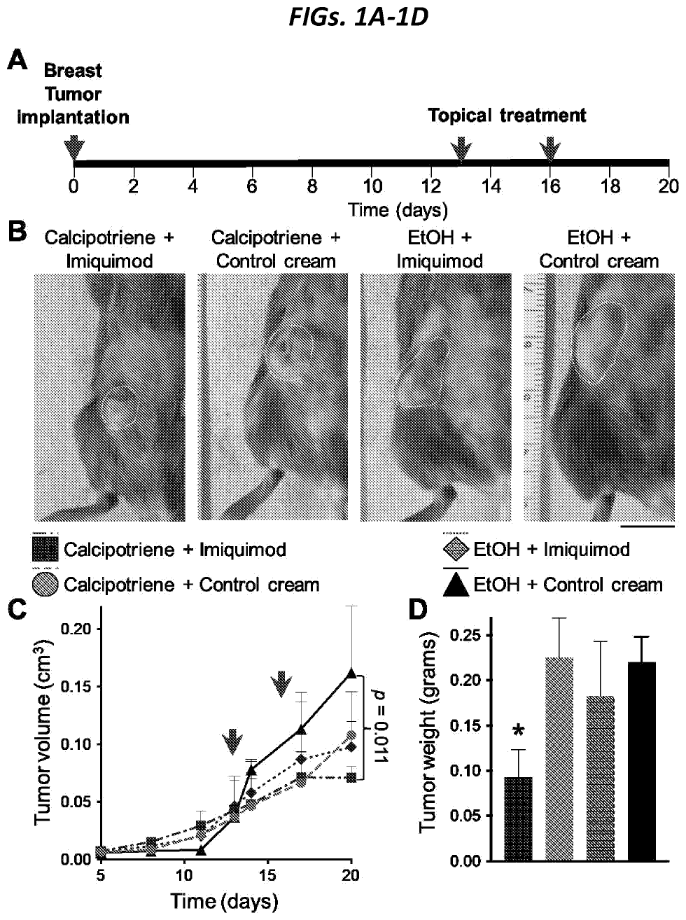

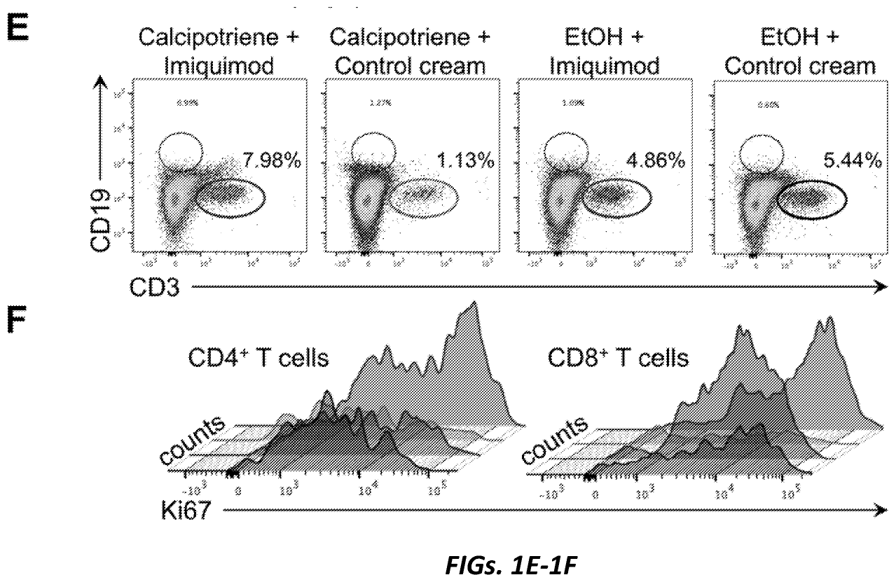

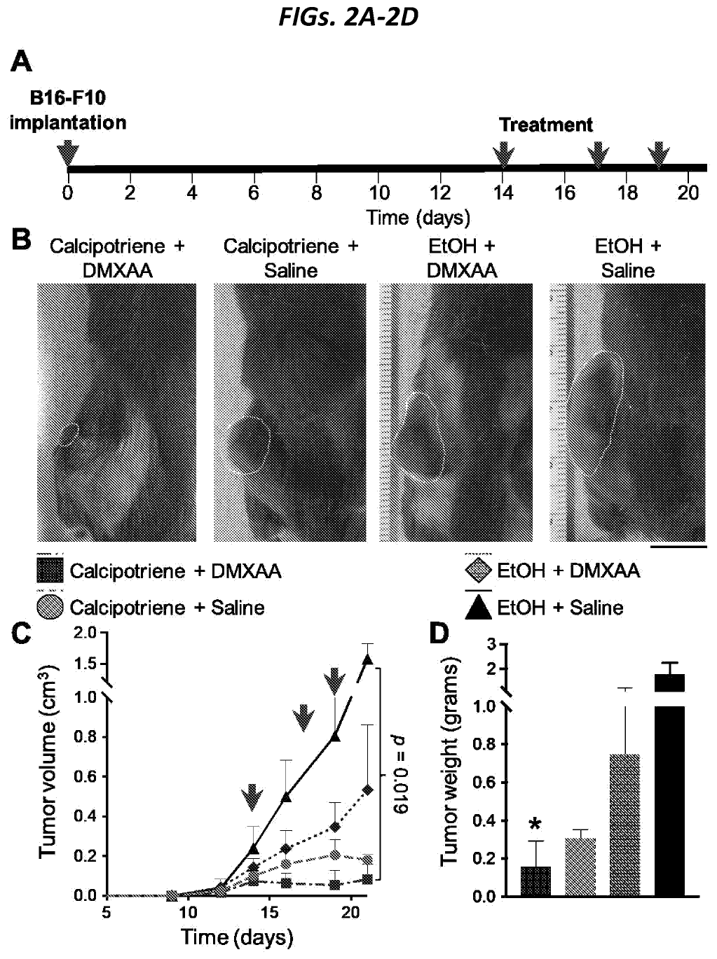

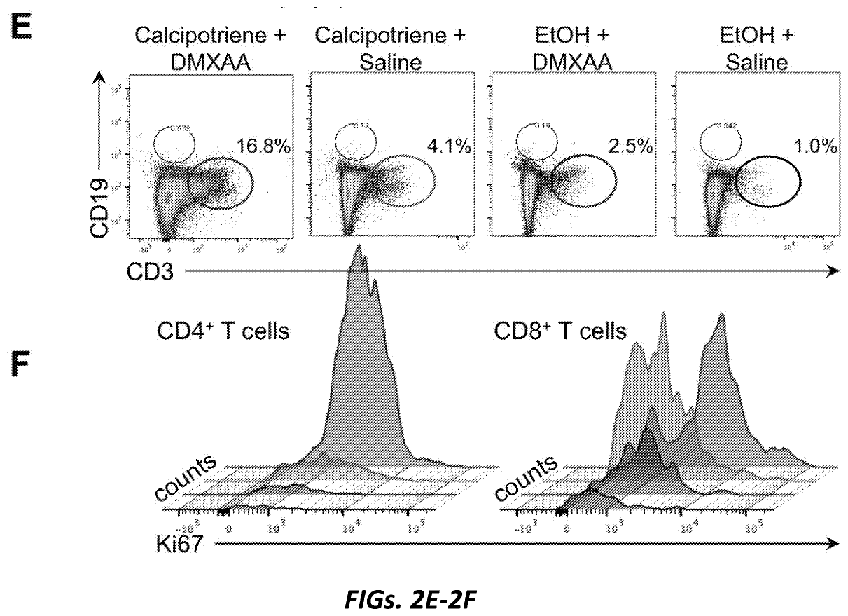

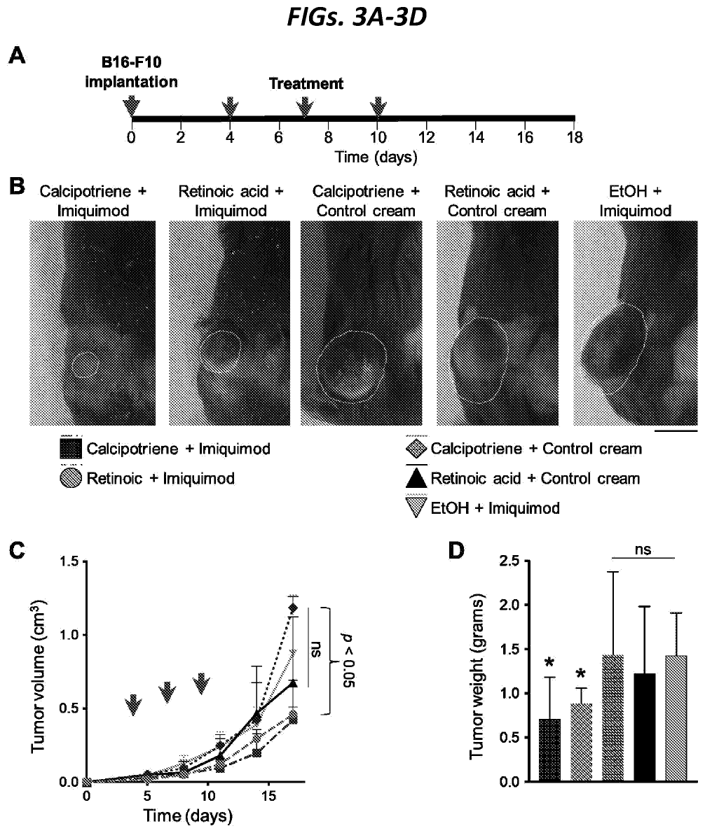

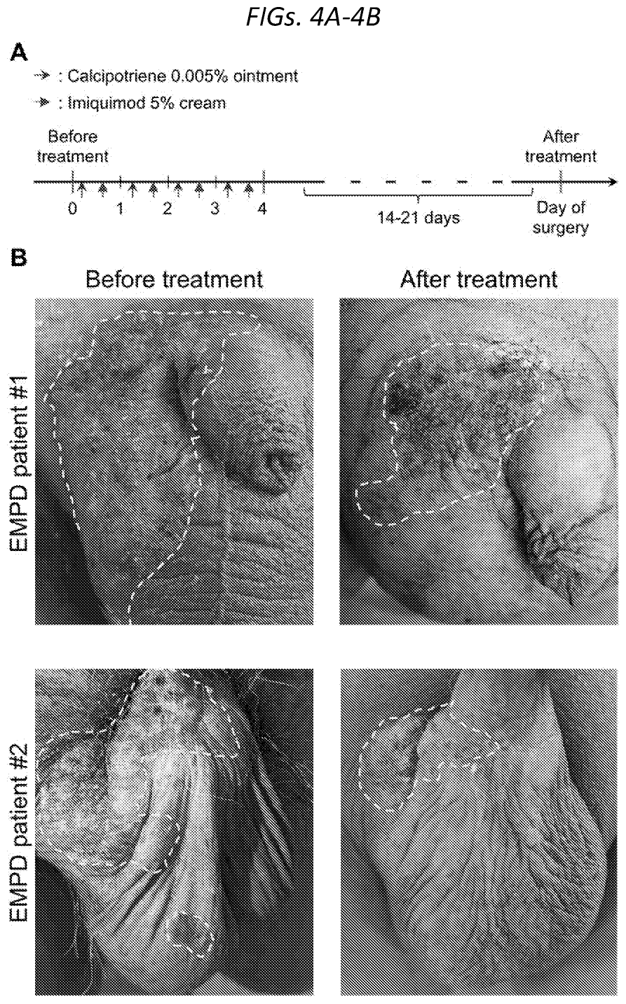

Breast cancer cells were obtained from PyMTtg mice that develop spontaneous breast cancer resembling luminal type of breast cancer in humans¹². Breast cancers of PyMTtg mice were harvested before the tumors reached 2 cm in diameter. Cancers were excised and dissociated into single cell suspension. Breast cancer cells were re-suspended at a concentration of 1×10⁶ cells per 100 ul of 1:1 ratio of RPMI and Matrigel Membrane Matrix (Corning). Inguinal regions of recipient mice were shaved prior to cancer cell injection. 1×10⁶ cells were injected subcutaneously at day 0. Topical treatments were applied when tumor sizes reached 5 mm in diameter. Mice were divided into 4 groups and were treated with either calcipotriene (80 nmol) or ethanol (EtOH) as carrier directly on the tumor sites. Following treatment of either calcipotriene or EtOH, tumor sites were also treated with topical application of either imiquimod 5% cream or moisturizing cream (control cream). Calcipotriene/EtOH and imiquimod/control cream treatments were re-applied one more time 3 days later (FIG. 1A). Animals were monitored daily and tumors measured over time to determine the impact of topical treatments on breast cancer growth. At harvest, tumors were weighed and the tumor infiltrating T cells were profiled using flow cytometry.

Two applications of calcipotriene plus imiquimod combination led to a significant inhibition of cutaneous breast tumor growth compared to EtOH plus control cream-treated breast tumors (p=0.011, FIGS. 1B-D). Either calcipotriene or imiquimod applied alone did not show a significant impact on breast cancer growth after two applications (FIG. 1B-D). Surprisingly, calcipotriene plus imiquimod combination caused a significant induction of T cell (CD3+CD19-) infiltrate within the treated tumors (FIG. 1E) and the proliferation (Ki67+) of CD4+ and CD8+ tumor infiltrating T lymphocytes (FIG. 1F). These findings highlight the role of antitumor immunity in mediating the effect of calcipotriene plus imiquimod treatment against breast cancer.

### Example 2. Calcipotriene Plus DMXAA Blocked Melanoma Progression

Melanoma Cells (B16-F10, ATCC, Manassas, Va.) were injected subcutaneously into flanks of mice at 2×10⁵ cells per 100 ul of 1:1 ratio of DMEM (Corning) at day 0. Flank regions of recipient mice were shaved prior to cancer cell injection.

Topical treatment with calcipotriene or EtOH together with intraperitoneal (IP) injection of DMXAA (murine STING agonist, 5,6-Dimethylxanthenone-4-acetic Acid, ASA404, or Vadimezan) or normal saline were initiated when tumor sizes reached 5 mm in diameter. Mice were divided into 4 groups and were treated topically with either calcipotriene (40 nmol) or ethanol (EtOH) as carrier directly on the tumor sites. Following treatment of either calcipotriene or EtOH (control), mice were injected IP with either 400 g DMXAA in 100 normal saline or normal saline alone (control). Calcipotriene/EtOH and DMXAA/saline treatments were re-applied two more times (FIG. 2A). Animals were monitored daily and tumors measured over time to determine the impact of treatments on melanoma growth. At harvest, tumors were weighed, and the tumor infiltrating T cells were profiled using flow cytometry.

Three applications of the calcipotriene plus DMXAA combination led to a significant inhibition of cutaneous melanoma growth compared to EtOH plus saline-treated melanoma (p=0.019, FIG. 2B-D). Calcipotriene alone showed efficacy in inhibiting melanoma growth but DMXAA alone did not show a significant impact on melanoma development after three applications (FIG. 2B-D). Calcipotriene plus DMXAA combination caused significant induction of T cell (CD3+CD19-) infiltrate within the treated tumors (FIG. 1E) and the proliferation (Ki67+) of CD4+ and CD8+ tumor infiltrating T lymphocytes (FIG. 1F), which was absent in calcipotriene alone, DMXAA alone and EtOH plus saline control groups. These findings demonstrate that antitumor immunity mediated the effect of calcipotriene plus DMXAA treatment against melanoma.

### Example 3. Calcipotriene Plus Imiquimod and Retinoic Acid Plus Imiquimod Suppressed Melanoma Growth

In a complementary set of experiments, B16-F10 melanoma cells were injected subcutaneously into flanks of mice. Topical treatments were applied when tumors became palpable. Mice were divided into 5 groups and were treated with either calcipotriene (20 nmol), retinoic acid (20 nmol) or ethanol (EtOH) as carrier directly on the tumor sites. Following treatment of either calcipotriene, retinoic acid or EtOH, tumor sites were also treated with topical application of either imiquimod 5% cream or moisturizing cream (control cream). Calcipotriene/retinoic acid/EtOH and imiquimod/control cream treatments were re-applied two additional times 3 days apart (FIG. 3A). Animals were monitored daily and tumors measured over time to determine the impact of topical treatments on melanoma growth. At harvest, tumors were photographed, and their weights were recorded.

Three applications of calcipotriene plus imiquimod and retinoic acid plus imiquimod combinations led to a significant inhibition of melanoma tumor growth compared to monotherapy with calcipotriene, retinoic acid or imiquimod as demonstrated by macroscopic images, tumor volume over time and tumor weights at harvest (p<0.05, FIG. 3B-D). These findings highlight the role of antitumor immunity in mediating the effect of calcipotriene plus imiquimod and retinoic acid plus imiquimod treatments against melanoma.

### Example 4. Calcipotriene and Imiquimod Combination Improves EMPD Lesions in Patients

To determine the efficacy of calcipotriene plus imiquimod combination in the treatment of EMPD in patients, we treated two male EMPD patients with topical calcipotriene 0.005% ointment in the morning and imiquimod 5% cream at night over 4 days before the surgical excision of the remaining lesions 14-21 days post treatment (FIG. 4A). The extent of the disease was documented by clinical images before treatment and at the time of the surgery, Interestingly, 4-day treatment with calcipotriene plus imiquimod combination led to a significant inflammation (i.e., immune activation) at the sites of EMPD lesions and the inflammation subsided over two weeks post treatment. Importantly, this treatment led to a significant reduction in the lesion size by the time of surgery, which was confirmed on the negative surgical margins of the reduced lesions that were excised (yellow dashed lines, FIG. 4B).

### REFERENCES

All patents, patent applications and publications mentioned in this specification are herein incorporated by reference to the same extent as if each independent patent and publication was specifically and individually indicated to be incorporated by reference.
1. Bichakjian C K, Halpern A C, Johnson™, Foote Hood A, Grichnik J M, Swetter S M, Tsao H, Barbosa V H, Chuang T Y, Duvic M, Ho V C, Sober A J, Beutner K R, Bhushan R, Smith Begolka W, American Academy of D. Guidelines of care for the management of primary cutaneous melanoma. American Academy of Dermatology. Journal of the American Academy of Dermatology 2011: 65; 1032-1047
2. Kanitakis J. Mammary and extramammary Paget's disease. J Eur Acad Dermatol Venereol 2007: 21; 581-590
3. Lookingbill D P, Spangler N, Helm K F. Cutaneous metastases in patients with metastatic carcinoma: a retrospective study of 4020 patients. Journal of the American Academy of Dermatology 1993: 29; 228-236
4. Adams S, Kozhaya L, Martiniuk F, Meng T C, Chiriboga L, Liebes L, Hochman T, Shuman N, Axelrod D, Speyer J, Novik Y, Tiersten A, Goldberg J D, Formenti S C, Bhardwaj N, Unutmaz D, Demaria S. Topical TLR7 agonist imiquimod can induce immune-mediated rejection of skin metastases in patients with breast cancer. Clin Cancer Res 2012: 18; 6748-6757
5. Salazar L G, Lu H, Reichow J L, Childs J S, Coveler A L, Higgins D M, Waisman J, Allison K H, Dang Y, Disis M L. Topical Imiquimod Plus Nab-paclitaxel for Breast Cancer Cutaneous Metastases: A Phase 2 Clinical Trial. JAMA Oncol 2017: 3; 969-973
6. Sendagorta E, Herranz P, Feito M, Ramirez P, Floristan U, Feltes R, Benito D M, Casado M. Successful treatment of three cases of primary extramammary Paget's disease of the vulva with Imiquimod—proposal of a therapeutic schedule. J Eur Acad Dermatol Venereol 2010: 24; 490-492
7. Demehri S, Turkoz A, Manivasagam S, Yockey L J, Turkoz M, Kopan R. Elevated epidermal thymic stromal lymphopoietin levels establish an antitumor environment in the skin. Cancer cell 2012: 22; 494-505
8. Demehri S, Cunningham T J, Manivasagam S, Ngo K H, Moradi Tuchayi S, Reddy R, Meyers M A, DeNardo D G, Yokoyama W M. Thymic stromal lymphopoietin blocks early stages of breast carcinogenesis. The Journal of clinical investigation 2016: 126; 1458-1470
9. Cunningham T J, Tabacchi M, Eliane J P, Tuchayi S M, Manivasagam S, Mirzaalian H, Turkoz A, Kopan R, Schaffer A, Saavedra A P, Wallendorf M, Cornelius L A, Demehri S. Randomized trial of calcipotriol combined with 5-fluorouracil for skin cancer precursor immunotherapy. The Journal of clinical investigation 2017: 127; 106-116
10. Ganti K P, Mukherji A, Surjit M, Li M, Chambon P. Similarities and differences in the transcriptional control of expression of the mouse TSLP gene in skin epidermis and intestinal epithelium. Proceedings of the National Academy of Sciences of the United States of America 2017
11. Weiss J M, Guerin M V, Regnier F, Renault G, Galy-Fauroux I, Vimeux L, Feuillet V, Peranzoni E, Thoreau M, Trautmann A, Bercovici N. The STING agonist DMXAA triggers a cooperation between T lymphocytes and myeloid cells that leads to tumor regression. Oncoimmunology 2017: 6; e1346765
12. Vargo-Gogola T, Rosen J M. Modelling breast cancer: one size does not fit all. Nature reviews. Cancer 2007: 7; 659-672
13. Tebes S, Cardosi R, Hoffman M. Paget's disease of the vulva. Am J Obstet Gynecol 2002: 187; 281-283; discussion 283-284
14. Hendi A, Brodland D G, Zitelli J A. Extramammary Paget's disease: surgical treatment with Mohs micrographic surgery. Journal of the American Academy of Dermatology 2004: 51; 767-773
15. Itonaga T, Nakayama H, Okubo M, Mikami R, Nogi S, Tajima Y, Sugahara S, Tokuuye K. Radiotherapy in patients with extramammary Paget's disease—our own experience and review of the literature. Oncol Res Treat 2014: 37; 18-22

1. A method for treating a proliferative disorder in the skin of a subject in need thereof, said method comprising administering a therapeutically effective amount of (i) an agent that activates T cells through TSLP induction and (ii) an innate immune cell activating agent, preferably directly to a site of proliferation in the skin of the subject, thereby treating the proliferative disorder in the skin of the subject.

2. The method of claim 1, wherein the proliferative disorder in the skin is a non-keratinocyte cancer.

3. The method of claim 2, wherein the non-keratinocyte cancer is melanoma, extramammary Paget's disease or a breast cancer metastasis.

4. The method of claim 1, wherein the site of proliferation in the skin is a tumor.

5. The method of claim 1, wherein the site of proliferation in the skin comprises a metastasis within the chest wall of the subject.

6. The method of claim 2, wherein the non-keratinocyte carcinoma is not a Squamous Cell Carcinoma (“SCC”), Basal Cell Carcinoma (“BCC”), or a keratinocyte cancer.

7. The method of claim 1, which does not include administering a cytotoxic agent.

8. The method of claims 1-8, wherein the agent that activates T cells through TSLP induction is calcipotriene or retinoic acid.

9. The method of claims 1-9, wherein the innate immune cell activating agent is a TLR agonist, STING agonist, or oncolytic virus.

10. The method of claim 9, wherein the TLR agonist is imiquimod, resiquimod, 852A, motolimod (VTX-2337); CpG-ODN; or ODN2395.

11. The method of claim 9, wherein the STING agonist is DMXAA, MK-1454 or ADU-S100.

12. The method of claim 9, wherein the oncolytic virus is Talimogene Laherparepvec (T-Vec).

13. The method of claim 1-12, wherein a composition comprising an agent that activates T cells through TSLP induction and a composition comprising an innate immune cell activating agent are administered concomitantly or sequentially.

14. The method of claim 1-12, wherein a single composition comprising an agent that activates T cells through TSLP induction and an innate immune cell activating agent is administered.

15. The method of claims 1-14, wherein one or both of the agent that activates T cells through TSLP induction and the innate immune cell activating agent is topically administered.

16. An agent that activates T cells through TSLP induction, and an innate immune cell activating agent method, for use in treating a proliferative disorder in the skin of a subject in need thereof, preferably in the absence of a cytotoxic agent.

17. The agent that activates T cells through TSLP induction, and the innate immune cell activating agent, for the use of claim 16, wherein the proliferative disorder in the skin is a non-keratinocyte cancer.

18. The agent that activates T cells through TSLP induction, and the innate immune cell activating agent, for the use of claim 17, wherein the non-keratinocyte cancer is melanoma, extramammary Paget's disease or a breast cancer metastasis.

19. The agent that activates T cells through TSLP induction, and the innate immune cell activating agent, for the use of claim 16, wherein the site of proliferative disorder in the skin is a tumor.

20. The agent that activates T cells through TSLP induction, and the innate immune cell activating agent, for the use of claim 16, wherein the proliferative disorder in the skin comprises a metastasis within the chest wall of the subject.

21. The agent that activates T cells through TSLP induction, and the innate immune cell activating agent, for the use of claim 17, wherein the non-keratinocyte carcinoma is not a Squamous Cell Carcinoma (“SCC”), Basal Cell Carcinoma (“BCC”), or a keratinocyte cancer.

22. The agent that activates T cells through TSLP induction, and the innate immune cell activating agent, for the use of claims 16-21, wherein the agent that activates T cells through TSLP induction is calcipotriene or retinoic acid.

23. The agent that activates T cells through TSLP induction, and the innate immune cell activating agent, for the use of claims 16-22, wherein the innate immune cell activating agent is a TLR agonist, STING agonist, or oncolytic virus.

24. The agent that activates T cells through TSLP induction, and the innate immune cell activating agent, for the use of claim 23, wherein the TLR agonist is imiquimod, resiquimod, 852A, motolimod (VTX-2337); CpG-ODN; or ODN2395.

25. The agent that activates T cells through TSLP induction, and the innate immune cell activating agent, for the use of claim 23, wherein the STING agonist is DMXAA, MK-1454 or ADU-S100.

26. The agent that activates T cells through TSLP induction, and the innate immune cell activating agent, for the use of claim 23, wherein the oncolytic virus is Talimogene Laherparepvec (T-Vec).

27. The agent that activates T cells through TSLP induction, and the innate immune cell activating agent, for the use of claims 16-26, wherein a composition comprising an agent that activates T cells through TSLP induction and a composition comprising an innate immune cell activating agent are administered concomitantly or sequentially.

28. The agent that activates T cells through TSLP induction, and the innate immune cell activating agent, for the use of claims 16-26, wherein a single comprising an agent that activates T cells through TSLP induction and an innate immune cell activating agent is administered.

29. The agent that activates T cells through TSLP induction, and the innate immune cell activating agent, for the use of claims 16-28, wherein one or both of the agent that activates T cells through TSLP induction and the innate immune cell activating agent is topically administered.
